(19) United States
(12) Patent Application Publication
(10) Pub. No.: US 2024/0090888 A1
(43) Pub. Date: Mar. 21, 2024

(54) Surgical Constructs and Methods of Tissue Repairs

(71) Applicant: Arthrex, Inc., Naples, FL (US)

(72) Inventors: Thomas DOONEY, Jr., Naples, FL (US); Shaun LEBLANC, Naples, FL (US); Derek C. SULLIVAN, Naples, FL (US); Trevor D. ARNOLD, Naples, FL (US)

(73) Assignee: Arthrex, Inc., Naples, FL (US)

(21) Appl. No.: 17/932,368

(22) Filed: Sep. 15, 2022

(51) Int. Cl. A61B 17/04; A61F 2/08

(52) U.S. Cl. CPC A61B 17/0401; A61F 2/0811; A61B 2017/0445; A61B 2017/0448; A61B 2017/0464; A61F 2002/0852

(57) ABSTRACT

Surgical constructs, assemblies, and methods for tissue repairs are disclosed. A surgical construct can include a fixation device with a sliding flexible coupler and a covering for the flexible coupler. The covering includes a hollow portion adjacent a tail portion. Limbs of the flexible coupler are secured within the hollow portion. The tail portion of the covering is passed through tissue such that the hollow portion along with the limbs of the sliding flexible coupler are passed through the tissue simultaneously.

### BACKGROUND

The disclosure relates to the field of surgery and, more specifically, to surgical constructs and tissue repairs for reconstructive surgeries.

### SUMMARY

Surgical constructs and methods of tissue repairs are disclosed.

A surgical construct can include a fixation device with a flexible coupler and a covering for the flexible coupler. A covering for the flexible coupler can include a hollow portion and a tail portion. Limbs of the flexible coupler are secured within the hollow portion. In use, the tail portion of the covering is passed through tissue such that the hollow portion along with the limbs of the flexible coupler are passed through the tissue. The covering can be released from the fixation device and removed from the limbs of the flexible coupler. A fixation device can be a suture anchor. A flexible coupler can be a sliding suture attached to a knotless suture anchor. A covering can be removably attached to and detached from the flexible coupler. A surgical construct can be knotless, self-locking, and tensionable.

A flexible coupler can extend between a suture anchor and a covering with a hollow passage that retains, holds, and covers limbs of the flexible coupler. The covering can consist essentially of suture. The covering can include a hollow passage terminating in a tail portion. Limbs of the flexible coupler can be securely retained within the hollow passage via friction. The hollow passage with the limbs of the flexible coupler can be pulled through soft tissue in one step. The covering can be a one-piece suture construct. The covering can be securely engaged to and disengaged from the flexible coupler. The suture anchor can provide at least one anchoring point of a first tissue affixed to a second tissue, for example, soft tissue to bone. The flexible coupler can be suture or tape.

Methods of knotless, tensionable tissue repairs are also disclosed. A surgical construct can provide knotless tissue to bone fixation, without knot formation and with increased fixation, eliminating suture shuttling steps. A tissue can be rotator cuff tendon.

### DESCRIPTION OF THE DRAWINGS

FIGS. 1-3 illustrate various views of a surgical construct.

FIGS. 4 and 5 illustrate various views of another surgical construct.

FIGS. 6 and 7 illustrate various views of another surgical construct.

FIGS. 8-12 illustrate steps of a tissue repair.

### DETAILED DESCRIPTION

The disclosure provides surgical systems, assemblies, constructs, and methods for tissue repairs and reconstructions.

A surgical construct can include a fixation device with a sliding suture and a suture covering (a suture sock). A suture covering includes a hollow suture portion and a tail portion. Limbs of the sliding suture are secured within the hollow suture portion via friction. In use, the tail of the covering is passed through tissue such that the hollow suture portion along with the limbs of the sliding suture are pulled through the tissue. The covering is released from the suture anchor and the covering is removed from the limbs of the sliding suture. The fixation device can be a suture anchor.

The covering can be manufactured as a one-piece hollow suture construct that can fit multiple limbs of suture. The end of the covering can taper down to a single limb, for easier suture management. The limb of the covering can be easily passed with standard suture passing instrumentation. The covering stays on the suture as a result of its snug fit, overall friction, and “Chinese finger trap” like function. A grasper can be used to pull up on the covering at the base of the suture anchor. This action “breaks the seal” and makes it easy to pull the covering off from the base or from the suture end.

The fixation device can be an anchor, button, implant, screw, plate, suture loop/button construct, or combinations thereof, that allow attachment to a sliding flexible coupler. The fixation device can be a knotless suture anchor such as the two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of all of which are fully incorporated by reference in their entirety herein. A knotless fixation device comprises an anchor body (or screw) and an eyelet.

Methods of forming knotless soft tissue repairs are also disclosed. An exemplary method of forming a knotless soft tissue repair comprises: (i) securing limbs of a flexible coupler (slidingly attached to a fixation device) to a suture covering; and (ii) passing the suture covering together with the limbs of the flexible coupler through soft tissue. The method can further include securing the fixation device to bone. The method can further include attaching the limbs to a hollow portion of the suture covering by friction; inserting the fixation device into a hole in bone; loading a tail of the suture covering into a suture passer; passing the suture covering with limbs of the flexible coupler through soft tissue; and removing the suture covering from the fixation device and from the limbs of the flexible coupler.

The flexible coupler can be any strand or fiber that can allow attachment of a first tissue to a second tissue, such as soft tissue to bone. The flexible coupler can be suture or suture tape, among many others. The fixation device can be a knotless suture anchor.

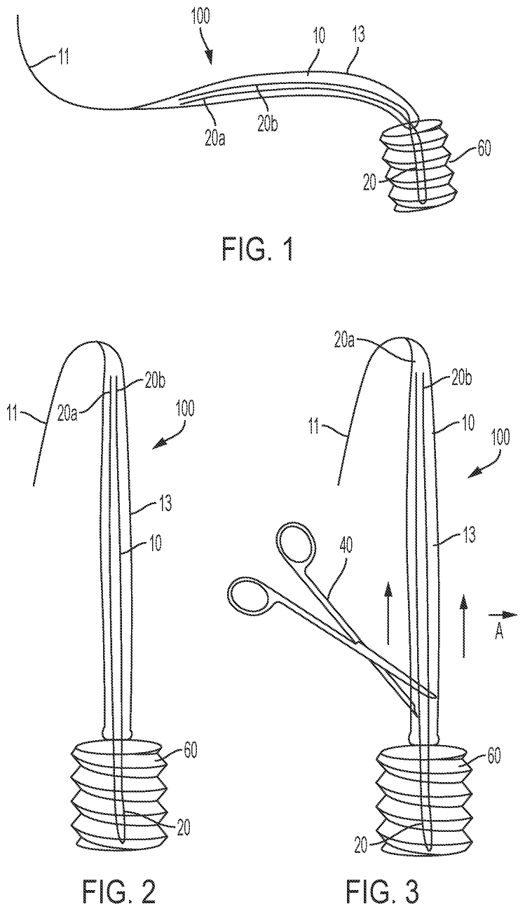

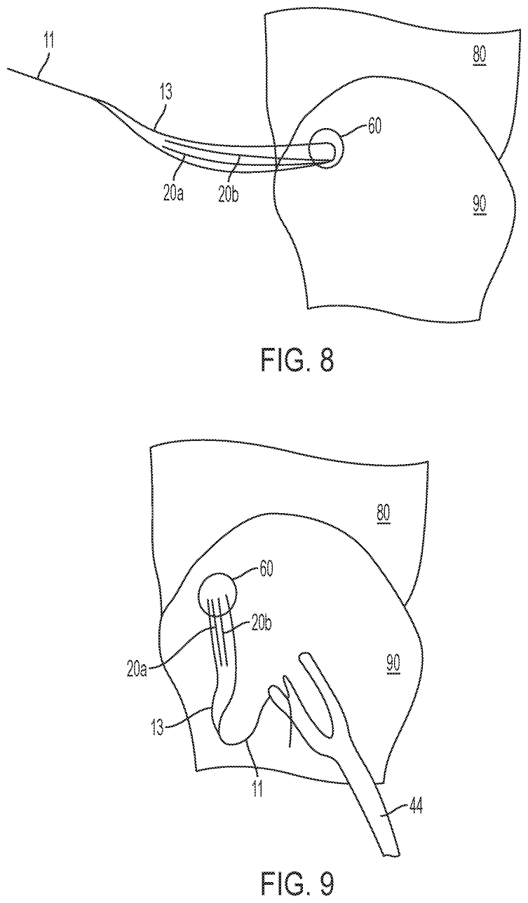

Referring now to the drawings, where like elements are designated by like reference numerals, FIGS. 1-7 illustrate exemplary surgical assemblies 100, 200, 300 including at least one surgical construct 10, 110, 210 (cover 10, 110, 210; covering 10, 110, 210; suture cover 10, 110, 210; suture covering 10, 110, 210; suture holder 10, 110, 210; suture retainer 10, 110, 210; suture sock 10, 110, 210; suture protector 10, 110, 210; suture shuttle 10, 110, 210) of the present disclosure. FIGS. 8-12 illustrate steps of an exemplary tissue repair 101 with surgical assemblies 100, 200, 300.

Surgical assembly 100 of FIGS. 1-3 includes exemplary construct 10 covering at least a portion of flexible coupler 20 slidably attached to fixation device 60. Construct 10 is a hollow suture construct that can fit multiple limbs of suture. As shown in FIGS. 1-3, construct 10 includes a hollow portion or hollow region 13 terminating in a single limb/tail 11. The end of the suture construct 10 tapers down to one manageable limb 11, preferably a #2 suture. As detailed below, this limb 11 is easily passed with standard suture passing instrumentation. Construct 10 stays on the flexible coupler 20 (suture 20) as a result of its snug fit, overall friction, and “Chinese finger trap” like function. Hollow region 13 of the construct 10 holds, retains, houses, covers and/or protects at least part of flexible coupler 20, for example, limbs 20a, 20b of flexible coupler 20. In an exemplary embodiment, hollow region 13 of construct 10 covers most of the length of limbs 20a, 20b located outside fixation device 60, for example, the whole lengths of limbs 20a, 20b located outside fixation device 60.

Construct 10 can be a one-piece construct. Construct 10 can be a one-piece suture construct. Construct 10 can be securely engaged to and disengaged from the flexible coupler 20. Construct 10 can be securely engaged to and disengaged from the fixation device 60. Fixation device 60 can be a suture anchor that provides at least one anchoring point of a first tissue affixed to a second tissue, for example, soft tissue to bone. Flexible coupler 20 can be suture or tape. Flexible coupler 20 can be attached to fixation device 60 by sliding through at least a portion of the fixation device 60, for example, by extending/sliding through at least a portion of a body of the fixation device 60.

Construct 10 can be securely engaged to and disengaged from the fixation device 60. As shown in FIG. 3, instrument 40 (for example, grasper 40) can be used to pull up on the construct 10 at the base of the fixation device 60 (anchor 60) in the direction of arrow A. This action “breaks the seal” and makes it easy to pull construct 10 off from the base or from the suture end.

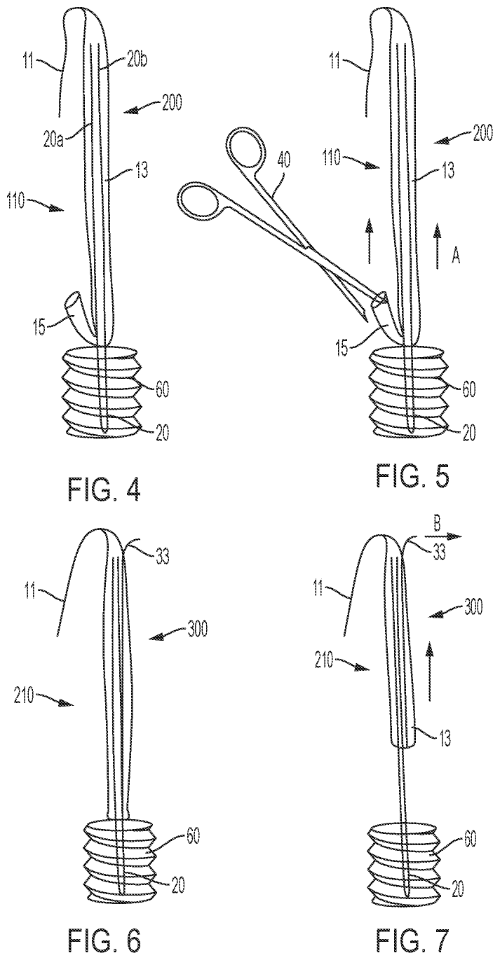

FIGS. 4 and 5 illustrate another exemplary embodiment of a surgical assembly 200 with construct 110 secured to fixation device 60. Construct 110 is about similar to construct 10 of FIGS. 1-3 in that hollow region 13 also covers most of limbs 20a, 20b of flexible coupler 20 extending out of the fixation device 60. However, construct 110 includes a tail 15 (“tail of sock” 15) located at the base of the fixation device. Flexible coupler 20 (limbs 20a, 20b) exits the construct 110 (suture sock 110) through body 13 of the construct 110 leaving a tail 15 at the base of the anchor 60. Tail 15 can be easily manipulated with instrument 40 (for example, grasper 40) to remove and disengage from the suture 20 by pulling in the direction of arrow A of FIG. 5.

In yet another embodiment, FIGS. 6 and 7 illustrate construct 210 as part of assembly 300. Construct 210 is about similar to constructs 10, 110 described above, but differs from them in that construct 210 includes a separate flexible strand 33 (release suture 33) that is incorporated into the hollow region 13. Flexible strand 33 pulls the construct up from the bottom, by pulling in the direction of arrow B (FIG. 7) to remove the construct. The release suture 33 can be pulled after the construct is passed through the tendon (soft tissue) to gain access to the previously contained sutures.

Reference is now made to FIGS. 8-12 which illustrate steps of an exemplary tissue repair 101 with the surgical assemblies and constructs of the present disclosure.

The exemplary tissue repairs detailed below are rotator cuff repairs. However, the disclosure is not limited to these exemplary-only embodiments and it must be understood that the present disclosure encompasses any tissue repair which involves side tissue of a repaired/reconstructed anatomical construct. Thus, surgical assemblies, constructs and repair methods detailed below have applicability not only to rotator cuff repairs, but also to surgical procedures such as Achilles tendon repair, patellar tendon repair, ACL/PCL reconstruction, hip and shoulder reconstruction procedures, and applications for suture used in or with suture anchors. The surgical constructs and repair methods of the present disclosure can be employed in tissue repairs that do not involve knot tying, for example, for use with suture anchors (such as PushLock® and/or SwiveLock® suture anchors) or for knotless arthroscopic suture repairs (such as knotless single row rotator cuff repair, or SpeedBridge™ repairs using no knots and only suture passing steps), among many others.

FIG. 8 illustrates a schematic rotator cuff 80 (tissue 80; soft tissue 80; rotator cuff tendon 80) secured over bone 90. FIG. 8 also illustrates placement of fixation device 60 of exemplary surgical assembly 100 including surgical construct 10. Fixation device 60 can be any of first and second medial fixation devices and first and second lateral fixation devices employed in a rotator cuff repair.

In an exemplary embodiment, fixation device 60 can be an anchor (knotted anchor, knotless anchor, or all-suture anchor), implant, button, screw or any fixation device that confers secure attachment and fixation of the rotator cuff 80 over bone 90 and that allow sliding of flexible strand 20 within a body of fixation device 60. The fixation device 60 can be a knotless suture anchor such as a two-piece Arthrex PushLock® anchor, disclosed in U.S. Pat. No. 7,329,272, or an Arthrex SwiveLock® anchor, disclosed in U.S. Pat. No. 8,012,174 and U.S. Pat. No. 9,005,246, the disclosures of both of which are fully incorporated by reference in their entirety herein.

At least one flexible coupler 20 can be incorporated with anchor placement. Flexible coupler 20 can be formed of any flexible material. In an embodiment, flexible coupler 20 is round suture. In an embodiment, flexible coupler 20 is FiberWire® suture. In an embodiment, flexible coupler 20 is a tape such as suture tape. In an embodiment, flexible coupler 20 is FiberTape® suture tape.

FIG. 9 illustrates tail 11 of construct 10 loaded into a suture passer 44 (suture passing instrument 44) such as an exemplary Arthrex Scorpion™ Suture Passer or Arthrex SutureLasso™.

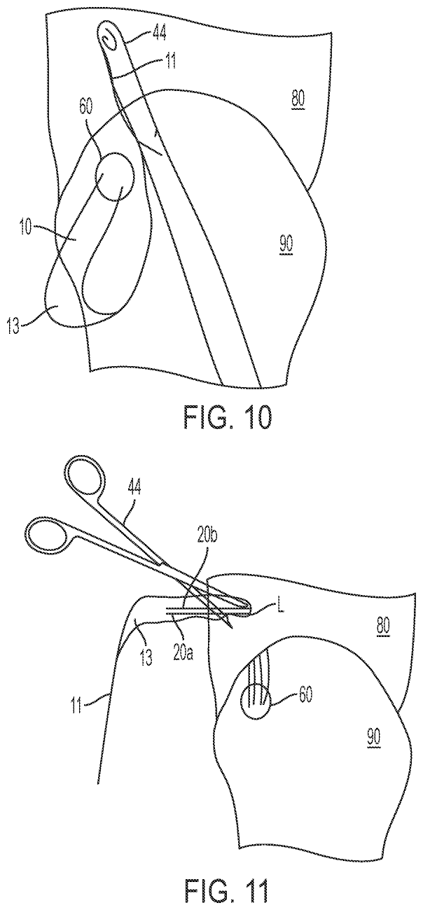

The tail is passed with the suture passer 44 (FIG. 10) at location “L” (FIG. 11).

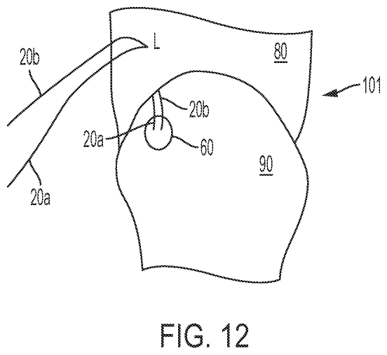

FIG. 11 illustrates passing of the construct 10 through soft tissue 80. A grasper can be used to loosen and remove the construct 10 (sock 10) from the limbs 20a, 20b and fixation device 60. FIG. 12 shows repair 101 with limbs 20a, 20b passed through soft tissue 80 at location L without using a shuttle link. Limbs 20a, 20b can be employed for additional suture passes to advance and/or complete repair of the rotator cuff, and as desired.

Reconstruction of the rotator cuff can be conducted by any method known in the art. In exemplary embodiments only, repairs of rotator cuff can be accomplished by repairs that do not involve knot tying, for example, with suture anchors (such as PushLock® and/or SwiveLock® suture anchors) or by knotless arthroscopic suture repairs (such as knotless single row rotator cuff repair, or SpeedBridge™ repairs using no knots and only suture passing steps), among many others.

An exemplary SutureBridge™ tendon repair technique, developed by Arthrex, Inc., and disclosed in U.S. Pat. No. 8,012,174 (the disclosure of which is herein incorporated by reference in its entirety) consists of a tied medial row constructed with two threaded suture anchors, combined with knotless lateral fixation using two Arthrex PushLocks® constructs. The construct enhances footprint compression and promotes tendon healing-to-bone with minimal knot tying.

An exemplary SpeedBridge™ technique, also developed by Arthrex, Inc., and disclosed in U.S. Pat. No. 9,005,246 (the entire disclosure of which is herein incorporated by reference) uses a threaded swivel anchor combined with FiberTape® suture tape to create a quick and secure SutureBridge™ construct with no knots and only two suture passing steps. In the SpeedBridge™ technique, a swivel anchor 60, preferably an Arthrex 4.75 mm SwiveLock® C 60, loaded with one strand of FiberTape® suture tape 20, is inserted into a medial bone socket. A suture cover such as construct 10, 110, 210 can be used to shuttle both FiberTape® tails 20a, 20b through the rotator cuff 80 simultaneously with construct 10, 110, 210. Tail 11 is passed through the rotator cuff 80 using a suture passing instrument such as the Scorpion™. The tails of the FiberTape® suture tape covered by hollow region 13 are shuttled through the rotator cuff 80. These steps are repeated for a second medial row anchor, or for any other suture passing steps, and as desired.

A method of double row fixation of tendon 80 to bone 90 to form a repaired rotator cuff as detailed in U.S. Pat. No. 8,012,174 comprises inter alia the steps of: (i) attaching first flexible strand 20 to a fixation device 60 (for example, a knotless fixation device or a soft anchor) and attaching second flexible strand 20 to a fixation device 60 (for example, a knotless fixation device or a soft anchor); (ii) securing the two fixation devices 60 into bone 90; (iii) securing limbs of the first flexible strand 20 to a hollow region 13 of construct 10, 110, 210 and passing limbs 20a, 20b of the first flexible strand 20 through soft tissue 80; and (iv) securing limbs 20a, 20b over the soft tissue 80. The method may further comprise the step of securing the fixation devices into a bone socket or tunnel or pushing the fixation devices into bone without forming a bone socket or tunnel. The method may further comprise the step of securing limbs of a second flexible strand to a hollow region 13 of construct 10, 110, 210 and passing limbs of the second flexible strand 20 through soft tissue 80.

Fixation device 60 can be a knotless suture anchor such as the two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of all of which are fully incorporated by reference in their entirety herein.

Exemplary fixations devices 60 can be two exemplary Arthrex SwiveLock® anchors 60 (for example, Arthrex 4.75-mm BioComposite SwiveLock® anchors) fixed to bone 90 (humerus 90). Fixation devices 60 can be employed to fixate soft tissue to bone by a SpeedFix™ or SpeedBridge™ configuration. The SpeedFix™ and SpeedBridge™ techniques, both developed by Arthrex, Inc., use a threaded swivel anchor, such as Arthrex SwiveLock® C anchor combined with FiberTape® (disclosed in U.S. Pat. No. 7,892,256) to create a quick and secure SpeedFix™ construct (a knotless single row repair) or a SpeedBridge™ construct (a knotless double row repair) with no knots and very few suture passing steps.

In the SpeedFix™ technique, FiberTape® suture is passed in an inverted mattress using a SutureLasso™ or Scorpion™ suture passer. The two suture limbs of the mattress stitch can then be inserted into the SwiveLock® anchor eyelet. The loaded eyelet is inserted into a prepared lateral bone socket until the anchor body contacts bone, and the tension is adjusted if necessary. The SwiveLock® C driver is rotated in a clockwise direction to complete the insertion. Using an open ended FiberWire® cutter, the FiberTape® tails are cut to complete the technique.

In an exemplary embodiment, reconstruction of tissue 80 by repairs 101 may be conducted by employing fixation devices, wherein at least one of the fixation devices is a soft anchor or an “all-suture” anchor. A soft anchor (soft suture anchor or all-suture soft knotless anchor) is provided with a soft anchor sleeve (sheath or tubular member) with two open ends, and at least two flexible shuttling strands extending through the soft anchor sleeve (sheath). The flexible strands may extend through the sleeve in similar or different directions and/or orientations and/or locations. The flexible tubular sleeve with the shuttling strands may be secured into or onto bone, and flexible strands may pass over soft tissue (rotator cuff) and are secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

A surgical assembly 100, 200, 300 for tissue reinforcement and repair comprises a flexible coupler 20; at least one fixation device 60 attached to the flexible coupler 20; and a surgical construct 10, 110, 210 holding/retaining/covering/protecting at least part of the flexible coupler 20. The fixation device 60 can be a knotless suture anchor. The tissue can be rotator cuff.

A method of soft tissue reconstruction comprises attaching soft tissue 80 to bone 90 with at least one flexible coupler 20 and without using a shuttling device. The method can further further comprise: slidingly attaching the at least one flexible coupler 20 to a fixation device 60; securing the fixation device 60 to bone 90; securely engaging tails 20a, 20b of the at least one flexible coupler 20 to a hollow region 13 of a suture covering 10, 110, 210, the suture covering terminating in a tail 11; passing the suture covering 10, 110, 210 and the tails 20a, 20b of the at least one flexible coupler 20 through the soft tissue 80; and disengaging the suture covering 10, 110, 210 from the flexible coupler 20. The at least one flexible coupler 20 is securely engaged to the suture covering 10, 110, 210 by friction. The suture covering 10, 110, 210 is a one-piece construct consisting essentially of suture. The fixation device 60 is a knotless anchor.

Flexible coupler 20 can be formed of any suture, tape, weave, fabric, ribbon, textile, web, or mesh, or any combinations of these materials. Flexible coupler 20 can be braided or multi-filament suture such as FiberTape® suture tape (as disclosed in U.S. Pat. No. 7,892,256, the disclosure of which is incorporated in its entirety herewith) or collagen tape, or wide “tape like” material, or combinations thereof. Flexible coupler 20 can be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Flexible coupler 20 can be formed of any material or combination of materials that can be provided on or through a rotator cuff, along a length thereon, and stitched/sutured to provide additional fixation when the rotator cuff sides are further secured. The flexible coupler 20 may be absorbable or non-absorbable.

Flexible coupler 20 can consist essentially of suture or suture material, or combination of suture and other materials such as long chain synthetic polymers like polyester and nylon, or materials such as PET, silk nylon or absorbable polymers, or coating materials (such as wax, silk, or silicone products), among many others. Flexible coupler 20 can consist of strands with cross-sections of various forms and geometries, including round, oval, rectangular, or flat, among others, or combinations of such forms and geometries. In an embodiment, at least one of flexible coupler 20 can be provided as a suture which is braided, knitted or woven.

Flexible coupler 20 can be also formed of a stiff material, or combination of stiff and flexible materials, depending on the intended application. Flexible coupler 20 can be also coated and/or provided in different colors. In an embodiment, parts (or all) of construct 88, 188, 288 can be coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) Or other coatings, to improve lubricity of the suture and/or tape, pliability, handleability or abrasion resistance, for example.

Flexible coupler 20 can be also provided with tinted tracing strands, or otherwise contrast visually with other parts of the construct, which remain a plain, solid color, or displays a different tracing pattern, for example. Various structural elements of the surgical constructs may be visually coded, making identification and handling of the suture legs simpler. Easy identification of suture in situ is advantageous in surgical procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A one-piece suture covering comprising a hollow region terminating in a single tail.

2. The one-piece suture covering of claim 1 consisting essentially of suture.

3. The one-piece suture covering of claim 1, wherein the hollow region is configured to hold and retain limbs of a flexible coupler by friction.

4. The one-piece suture covering of claim 3, wherein the single tail is passed with the limbs of the flexible coupler through soft tissue in a single step and without shuttling instrumentation.

5. The one-piece suture covering of claim 3, wherein the limbs of the flexible coupler are slidingly attached to a fixation device.

6. The one-piece suture covering of claim 5, wherein the fixation device is a suture anchor and the flexible coupler is a suture or suture tape.

7. The one-piece suture covering of claim 6, wherein the suture is a high-strength suture.

8. A surgical assembly for soft tissue repairs, comprising:
a flexible coupler attached to a fixation device; and
a covering secured to limbs of the flexible coupler.

9. The surgical assembly of claim 8, wherein the covering is a one-piece suture construct comprising a hollow portion and a tail extending from the hollow portion.

10. The surgical assembly of claim 9, wherein limbs of the flexible coupler are secured to the hollow portion of the covering by friction.

11. The surgical assembly of claim 10, wherein the limbs of the flexible coupler are engaged to, and disengaged from, the hollow portion of the covering.

12. The surgical assembly of claim 10, wherein the limbs of the flexible coupler and the covering are passed simultaneously through soft tissue.

13. The surgical assembly of claim 9, wherein the tail is a #2 high-strength suture.

14. The surgical assembly of claim 8, wherein the flexible coupler is a suture or suture tape slidingly attached to the fixation device.

15. The surgical assembly of claim 8, wherein the flexible coupler is a high-strength suture and wherein the fixation device is a knotless suture anchor.

16. The surgical assembly of claim 8, wherein the fixation device is an anchor inserted into bone.

17. A method of soft tissue reconstruction comprising attaching soft tissue to bone with at least one flexible coupler and without using a shuttling device.

18. The method of claim 17, further comprising:
slidingly attaching the at least one flexible coupler to a fixation device;
securing the fixation device to the bone;
securely engaging tails of the at least one flexible coupler to a hollow region of a suture covering, the suture covering terminating in a tail;
passing the suture covering and the tails of the at least one flexible coupler through the soft tissue; and
disengaging the suture covering from the flexible coupler.

19. The method of claim 18, wherein the at least one flexible coupler is securely engaged to the suture covering by friction.

20. The method of claim 18, wherein the suture covering is a one-piece construct consisting essentially of suture.

21. The method of claim 18, wherein the fixation device is a knotless anchor.

22. The method of claim 18, wherein the soft tissue is rotator cuff and the fixation device is a lateral or medial fixation device for rotator cuff repair.

23. The method of claim 18, wherein the soft tissue is rotator cuff and the method further comprises:
securing the at least one flexible coupler to a fixation device;
securing the fixation device at a first location in the bone;
attaching, by friction, limbs of the at least one flexible coupler to a hollow region of the suture covering;
passing the suture covering with the limbs through the rotator cuff; and
removing the suture covering from the limbs.

24. The method of claim 23, further comprising:
passing the limbs over the rotator cuff; and
attaching the limbs to at least another fixation device.

25. The method of claim 24, wherein the fixation device is a knotless anchor.

26. The method of claim 24, wherein the at least another fixation device is a knotless anchor.
